(19) United States
(12) Patent Application Publication
(10) Pub. No.: US 2022/0273872 A1
(43) Pub. Date: Sep. 1, 2022

(54) INTEGRATION OF SENSORS FOR DRUG DELIVERY COMPENSATION IN AUTOMATED MEDICATION DELIVERY (AMD) SYSTEMS

(71) Applicant: Insulet Corporation, Acton, MA (US)

(72) Inventors: Rangarajan NARAYANASWAMI, Weston, MA (US); Daniel ALLIS, Boxford, MA (US)

(21) Appl. No.: 17/668,124

(22) Filed: Feb. 9, 2022

Related U.S. Application Data
(60) Provisional application No. 63/154,179, filed on Feb. 26, 2021.

(51) Int. Cl. A61M 5/172; A61M 5/142; A61B 5/024; A61B 5/1455

(52) U.S. Cl. CPC A61M 5/1723; A61M 5/14244; A61B 5/024; A61B 5/14551; A61M 2230/201; A61M 2205/52; A61B 2562/0219; A61M 2205/3584; A61M 2205/3569

(57) ABSTRACT

Disclosed are techniques, products, and an automatic medication delivery system that includes a drug delivery system configured to implement a diabetes treatment management plan. The drug delivery system of the automatic medication delivery system includes a drug delivery device having a processor, a memory storing programming code executable by the processor, a drug container configured to contain a liquid drug, a pump drive mechanism configured to expel the liquid drug from the drug delivery device, and an auxiliary device interface coupled to the processor and configured with a data connection. The drug delivery system also includes a sensor module configured to couple to the auxiliary device interface of the drug delivery device, the sensor module including one or more sensors. The one or more sensors may be configured to detect movement or a physical attribute of a person wearing the drug delivery device and sensor.

### RELATED APPLICATIONS

This application claims the benefit of U.S. Provisional Patent Application No. 63/154,179, filed Feb. 26, 2021, the content of which is incorporated herein by reference in its entirety.

### BACKGROUND

In a non-diabetic person, insulin acts to allow the sugars consumed (in the form of carbohydrates) by the non-diabetic person to be provided to person's body cells and thereby reduce the amount of sugar in the person's body. The body cells convert the sugars to energy. After a meal, excess glucose may be stored in the liver of the person.

In contrast, the blood glucose levels of a diabetic continue to rise after consumption of a meal because there is not enough insulin to move the glucose into the body's cells of the diabetic. Persons with Type II diabetes do not use insulin efficiently (insulin resistance) and/or do not produce enough insulin (insulin deficiency), while persons with Type I diabetes make little or no insulin. Untreated, high blood glucose can eventually lead to complications such as blindness, nerve damage and kidney damage.

As such, blood glucose control in Type I and Type II diabetic patients remains a challenging problem. Closed loop automated medication delivery systems, such as automatic insulin dosing systems are currently a promising solution for the control of blood glucose, minimizing the number of hypoglycemic events and providing an acceptable time in range for the blood glucose. The automated medical delivery systems may be informed with current blood glucose levels and trends to drive the automated medication delivery system. Challenges remain because of variabilities in medication sensitivity and blunting of counter regulatory hormones arising from ongoing exercise as well as exercise several hours ago. It is well known that glucose uptake into skeletal muscle can persist for several hours after exercise. Consequently, there is a significant risk of exercise induced hypoglycemia. Further, the type of exercise, such as aerobic versus anaerobic exercise, has implications in blood glucose control.

While fitness devices worn at the wrist may provide information related to exercise, the quality and reliability of the data obtained by sensors located at the wrist of a user is not as good as the data quality and reliability obtained from sensors located at the core, upper arm or upper leg of a user.

In addition, wrist worn fitness devices often rely on wireless communications with another device, which may or may not be present when a user is participating in exercise. Therefore, real-time or substantially real-time, modification of drug delivery is delayed until the cessation of the exercise session. Furthermore, if the wrist worn fitness device is not worn either inadvertently or intentionally, insulin delivery cannot be controlled based on performed exercise.

It would be beneficial if a determination of whether a person with diabetes is participating in exercise so delivery of insulin by an automated drug delivery device may be more closely controlled and regulated.

### BRIEF SUMMARY

In one aspect, a drug delivery system, includes a drug delivery device including a processor, a memory storing programming code executable by the processor, a drug container configured to contain a liquid drug, a pump drive mechanism configured to expel the liquid drug from the drug delivery device, and an auxiliary device interface coupled to the processor and configured with a data connection. The drug delivery system also includes a sensor module configured to couple to the auxiliary device interface of the drug delivery device, the sensor module including one or more sensors, where the one or more sensors are respectively configured to measure parameters related to an orientation and a movement of the sensor module and a physical attribute of a user, and output a signal related to the measured parameters to the auxiliary device interface.

In another aspect, a method, includes receiving signals from a sensor module coupled to a wearer, where the signals are from an accelerometer, a gyroscope, and a heart rate sensor, the heart rate sensor is configured to detect a heart rate of the wearer, determining whether the received signals indicate the wearer is participating in physical activity, where physical activity is indicated when the received signals match within a preset threshold exercise indication information, and in response to determination that exercise has occurred, modifying an amount of a drug dosage to be delivered to the wearer. The method also includes outputting an actuation signal to a pump mechanism, where the actuation signal indicated the modified amount of the drug dosage.

### BRIEF DESCRIPTION OF THE DRAWINGS

To easily identify the discussion of any particular element or act, the most significant digit or digits in a reference number refer to the figure number in which that element is first introduced.

FIG. 1A illustrates an isometric view of an example of a drug delivery system including a sensor module and a drug delivery device.

FIG. 1B illustrates a top view of another example of the drug delivery system in accordance with another aspect of the disclosed subject matter.

FIG. 2 illustrates an isometric view of another example drug delivery system including a sensor module and a drug delivery device.

FIG. 3 illustrates a cross-sectional view of a further example of a drug delivery system including a sensor module and a drug delivery device.

FIG. 4 illustrates a cross-sectional view of another example of a drug delivery system including a sensor module and a drug delivery device.

FIG. 5A illustrates an example of a sensor module in accordance with one aspect of the disclosed subject matter.

FIG. 5B illustrates another example of a sensor module in accordance with another aspect of the disclosed subject matter.

FIG. 5C illustrates yet a further example of a sensor module in accordance with yet another aspect of the disclosed subject matter.

FIG. 6A illustrates a bottom view of an example of a sensor module within a drug delivery device in an aspect of a drug delivery system.

FIG. 6B illustrates a bottom view of another example of a sensor module within a drug delivery device in an aspect of a drug delivery system.

FIG. 6C illustrates a bottom view of yet another example of a sensor module within a drug delivery device in an aspect of a drug delivery system.

FIG. 7A illustrates an aspect of the subject matter in accordance with one embodiment.

FIG. 7B illustrates an aspect of the subject matter in accordance with one embodiment.

FIG. 8 illustrates an example of an automatic medication delivery system incorporating an example of the drug delivery system in accordance with another aspect of the disclosed subject matter.

FIG. 9 illustrates a process 900 in accordance with an embodiment.

### DETAILED DESCRIPTION

Different strategies for utilizing exercise information (once detected) may include prolonged predominantly aerobic exercise, temporary basal reduction, carbohydrate intake during/after exercise, sprint after exercise, nocturnal basal reduction to avoid a risk of hypoglycemia.

Diabetics when participating in brief intense exercise, predominantly anaerobic in nature, might need one or more of the following responses to the exercise: increased basal delivery of insulin to prevent/treat hyperglycemia (as a consequence of hepatic glucose production), an aerobic cool down (to mitigate ongoing hyperglycemia), carbohydrate intake after exercise to reduce the risk of delayed hypoglycemia and for recovery (if no observed hyperglycemia, right after the exercise) with a reduced insulin-to-carbohydrate ratio. In contrast to prolonged aerobic exercise, no nocturnal basal reduction may be needed in response to the brief intense anaerobic exercise.

The drug delivery system disclosed herein is described with reference to the examples illustrated in the drawings. The drug delivery system may be an element or a component within a larger automatic medication delivery system. In the examples, the drug delivery system may include a drug delivery device and a sensor module as well as other components that are described throughout the specification. In different aspects, the drug delivery system is a wearable drug delivery system that is configured to be worn by a user.

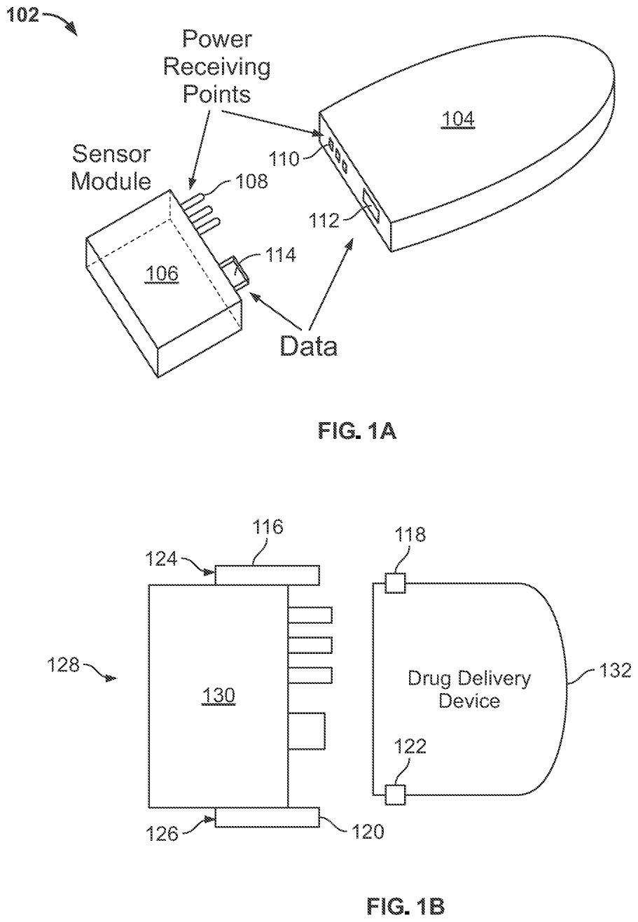

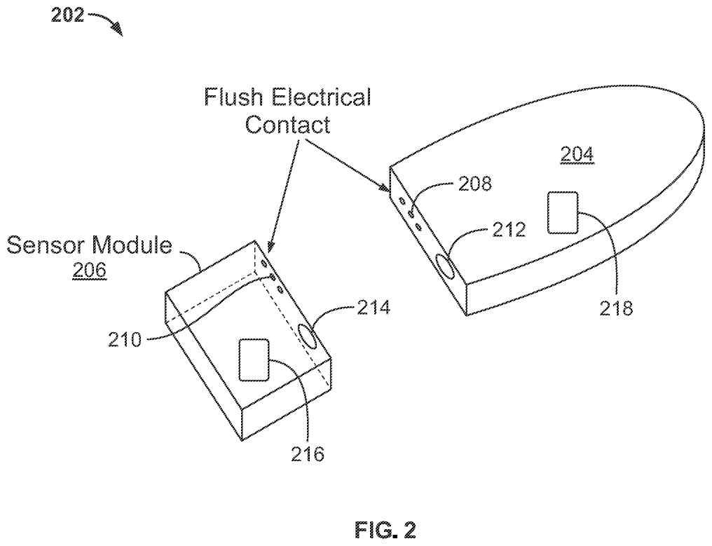

FIG. 1A illustrates an isometric view of an example of a drug delivery system including a sensor module 106 and a drug delivery device 104. The drug delivery system 102 includes a drug delivery device 104 and a sensor module 106. The drug delivery device 104 further includes power contacts 110 and a data connection 112. The sensor module 106 may include power receiving contacts 108 and data transfer connection 114. The power receiving contacts 108 of the sensor module 106 may connect to the power contacts 110 via a snap fit, compression fit, or the like. Similarly, the data transfer connection 114 may couple to the data connection 112 via a snap fit, compression fit, or the like. The data transfer connection 114 may be a connector such as universal serial bus (USB) connector, a micro-USB connector or the like. In addition, the power receiving contacts 108 and the data transfer connection 114 may be combined in one connector.

When the sensor module 106 is coupled to the drug delivery device 104, power may be delivered to the sensor module 106 via the connection of the power receiving contacts 108 to the power contacts 110. The sensor module 106 may include several different sensors that may be configured to detect different conditions or physical attributes of a wearer of the drug delivery system 102.

FIG. 1B illustrates a top view of another example of the drug delivery system in accordance with another aspect of the disclosed subject matter. In the example of FIG. 1B, the drug delivery system 128 includes a sensor module 130 and a drug delivery device 132. The sensor module 130 includes clip 116 and clip 120 that are configured to engage clip attachment 118 and clip attachment 122, respectively. The clips 116 and 120 may snap fit to the clip attachments 118 and 122 and thereby secure the sensor module 130 in place against the drug delivery device 132. The sensor module 130 may be released from the drug delivery device 132 by “unclipping” or unfastening the clips 116 and 120 from their respective clip attachments 118 and 122. For example, a user may engage both the release point 124 of clip 116 and the release point 126 of clip 120, which may cause, respectively, the end of the clip 116 opposite from the release point 124 to disengage from the clip attachment 118 as well as engage end of the clip 120 opposite from the release point 126 to disengage from the clip attachment 122. With the clips 116 and 120 disengaged sensor module 130 may be uncoupled from the drug delivery device 132.

While the clips 116 and 120 and their clip attachments 118 and 122 are shown other forms of snap fittings may be used to secure the sensor module 130 to the drug delivery device 132.

Another example of securing a sensor module to a drug delivery device may include magnetic attachment. FIG. 2 illustrates an isometric view of another example drug delivery system including a sensor module and a drug delivery device. In the example drug delivery system 202, a sensor module 206 may be configured to magnetically couple to the drug delivery device 204. The sensor module 206 may include electrical contacts 210, a magnet 214 and a communication device 216, which enables a wireless data connection, such as a Bluetooth® connection or the like. The drug delivery device 204 may include electrical power contacts 208, a magnet 212 and a communication device 218. The electrical power contacts 208 may be flush with an external surface of the drug delivery device 204 and the electrical contacts 210 may be flush with an external surface of the sensor module 206. The sensor module 206 and the drug delivery device 204 may be configured to couple to one another via magnets 214 and 212. The magnet 212 of the drug delivery device 204 may maintain contact with magnet 214 of the sensor module 206, which draws the electrical contacts 210 into alignment and electrical contact with electrical power contacts 208. Data from the sensor module 206 may be transferred via the communication device 216 to the drug delivery device 204, which receives the transferred data via the communication device 218.

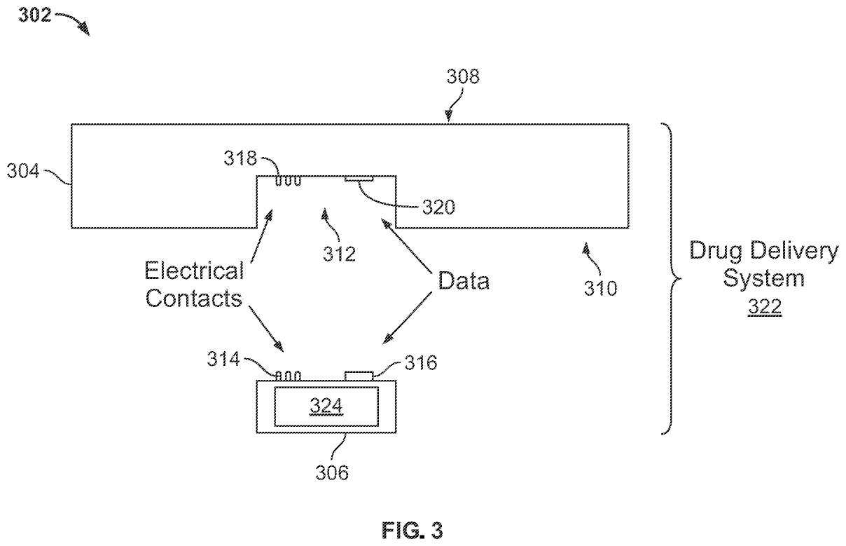

FIG. 3 illustrates a cross-sectional view of a further example of a drug delivery system including a sensor module and a drug delivery device.

In this example, the drug delivery system 322 includes a drug delivery device 302 and a sensor module 306. The drug delivery device 302 and the sensor module 306 may connect to one another utilizing a snap-fit mechanical coupling, such as the clip attachment 118 and clip 116 described in the earlier examples. However, for ease of illustration, the snap-fit mechanical coupling is not shown in this figure. The drug delivery device 302 may include a drug delivery device housing 304 having a housing top surface 308 and a housing bottom surface 310. The housing bottom surface 310 has a module opening 312 configured to hold the sensor module 306. The sensor module 306 may include electrical contacts 314, data connection 316 as well as sensors 324 (described with reference to another example). The sensors may be configured to generate data that is transferred from the sensor module 306 to the drug delivery device 302.

Not shown in this example is an adhesive layer that is configured to adhere the drug delivery device 302 to a surface (such as skin) and maintain the drug delivery housing 304 in contact with the surface (not shown), when adhered to the surface (i.e., the skin of the wearer). The sensor module 306 when held in the opening 312 contacts the surface to detect physical attributes, such as heart rate, blood oxygen saturation, perspiration, skin conductance, and other attributes. In addition, the sensors 324 in the sensor module 306 may also be configured to measure movements of the wearer using other sensors, such as an accelerometer, gyroscope and the like, that are collectively referred to as the sensors 324.

The sensor module 306 may connect to the drug delivery device 302 via electrical contacts 314 and the data connection 316. The sensor module 306 may receive electrical power from a power source (shown in another example) within the drug delivery device 302 by connecting the electrical contacts 314 to the power contacts 318. The data generated from the measurements made by the sensors 324 of the sensor module 306 may be transferred via a coupling of the data connection 316 with the data connection 320 of the drug delivery device 302. For example, the data related to the physical attributes and movements of the wearer generated by the sensors 324 by the sensor module 306 may be transferred to the drug delivery device 302, which may process the received data as described with reference to other examples.

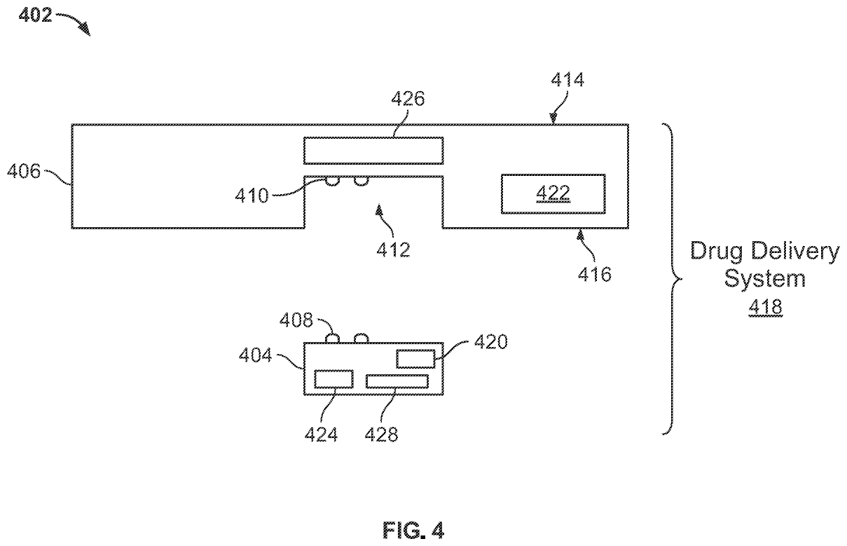

FIG. 4 illustrates a cross-sectional view of another example of a drug delivery system 418 including a sensor module 404 and a drug delivery device 402.

In the example, the drug delivery device 402 may include a delivery device communication circuitry 422, power contacts 410, a magnet 426, a drug delivery device housing 406 having a housing top surface 414 and a housing bottom surface 416, and a module opening 412. The module opening 412 is in the housing bottom surface 416 and is configured to hold the sensor module 404. The service module 404 may include electrical contacts 408, sensor communication circuitry 420, magnet 424 as well as sensors 428, which may include an accelerometer, a gyroscope, a perspiration detection sensor (e.g., a skin conductance detector), skin temperature, a heart rate monitor, blood oxygen sensor, and the like. Note the respective individual sensors may be separate within the sensor module 404 but are collectively referred to as sensors 428.

Not shown in this example is an adhesive layer that is configured to adhere the drug delivery device 402 to a surface (such as skin, not shown in this example) and maintain the drug delivery housing 406 in contact with the surface, when adhered to the surface (i.e., the skin of the wearer). The magnet 424 of sensor module 404 may magnetically couple to magnet 426 of the drug delivery system 402 and secure the sensor module 404 in the module opening 412. The module opening 412 in the drug delivery system 402 is configured to accept the sensor module 404 and be held in place by the attraction between magnet 426 and magnet 424. The sensor module 404 when held in the module opening 412 contacts the surface to detect physical attributes, such as heart rate, blood oxygen saturation, perspiration, and other attributes. In addition, the sensors 428 of the sensor module 404 may be configured to measure movements of the wearer using other sensors, such as an accelerometer, gyroscope and the like.

The sensor module 404 may electrically connect via electrical contacts 408 to the power contacts 410 of the drug delivery device 402 to obtain power. The sensor module 404 receive electrical power from a power source (shown in another example) within the drug delivery device 402 by electrically connecting the electrical contacts 408 to the power contacts 410. The power contacts 410 and electrical contacts 408 may be flush with the surface of the drug delivery device 402 and the sensor module 404, respectively.

The data generated from the measurements made by the sensors in the sensor module 404 may be transferred via a wireless communication link established by a pairing protocol between the sensor communication circuitry 420 of the sensor module 404 and the delivery device communication circuitry 422 of the drug delivery device 402. For example, the data related to the physical attributes and movements of the wearer made by the sensor module 404 may be wirelessly transferred to the drug delivery device 402, which may process the received data as described with reference to other examples.

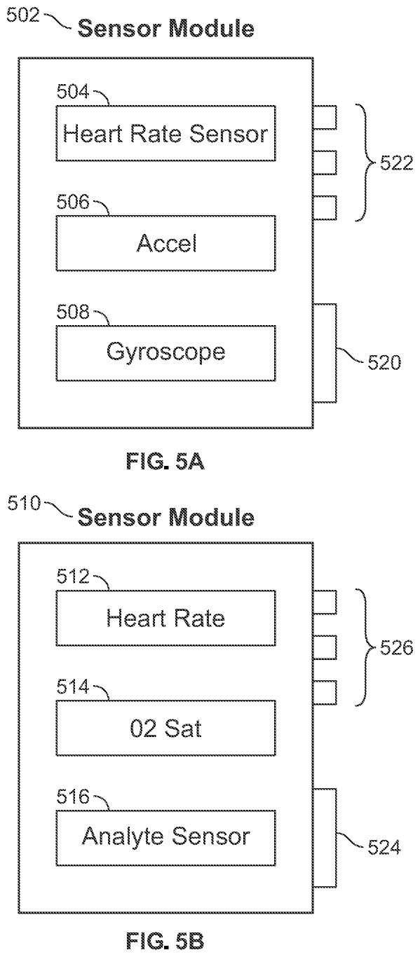

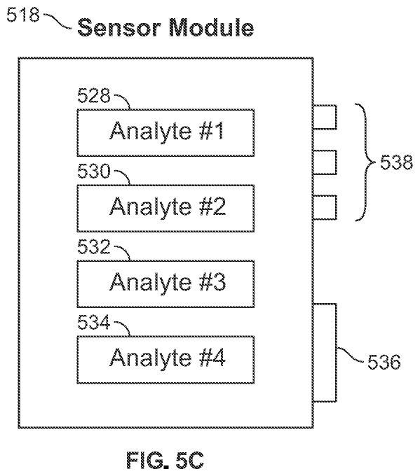

FIG. 5A illustrates an example of a sensor module in accordance with one aspect of the disclosed subject matter.

In this example, the sensor module 502 in an example drug delivery system may include one or more sensors. The one or more sensors, in this example, may include a heart rate sensor 504, an accelerometer (Accel) 506, and a gyroscope 508. Each of the respective sensors may generate data specific to the type of sensor. For example, the heart rate sensor 504 may generate data of the heart rate of the wearer of the drug delivery system based on physical indications of a heart rate of a wearer of the drug delivery system, the Accel 506 may generate data based on movement detected by the Accel 506. Similarly, the gyroscope 508 is configured to generate data based on inputs detected by the gyroscope 508. The data generated by the heart rate sensor 504. Accel 506 and the gyroscope 508 may be output via a data connection 520 for receipt by the drug delivery device (shown in other examples). The electrical connections 522 may receive electrical power from power contacts of a drug delivery device as shown in other examples. Other technical features may be readily apparent to one skilled in the art from the following figures, descriptions, and claims.

FIG. 5B illustrates another example of a sensor module in accordance with another aspect of the disclosed subject matter.

In this example, the sensor module 510 in an example drug delivery system may include one or more sensors. The one or more sensors, in this example, may include a heart rate sensor 512, oxygen (O₂) saturation (Sat) 514, and an analyte sensor 516. Each of the respective sensors may generate data specific to the type of sensor. For example, the heart rate sensor 512 may generate data of the heart rate of the wearer of the drug delivery system based on physical indications of a heart rate of a wearer of the drug delivery system, the oxygen (02) saturation (Sat) 514 may generate data based blood oxygen saturation measurements made from a wearer of the drug delivery system. Similarly, the gyroscope 508 is configured to generate data based on inputs detected by the gyroscope 508. The data generated by the heart rate sensor 504. Accel 506 and the gyroscope 508 may be output via a data connection 520. The electrical connections 526 may receive electrical power from power contacts of a drug delivery device as shown in other examples. Other technical features may be readily apparent to one skilled in the art from the following figures, descriptions, and claims.

FIG. 5C illustrates yet a further example of a sensor module in accordance with yet another aspect of the disclosed subject matter.

In this example, the sensor module 518 in an example drug delivery system may include one or more sensors. The one or more sensors, in this example, may include an analyte #1 528, analyte #2 530, analyte #3 532 and an analyte #4 534. Each of the respective analyte sensors 528, 530, 532 and 534 may generate data specific to a respective analyte, such as blood glucose, proteins, hormones or the like. For example, the analyte #1 528 may generate data related to a blood glucose measurement of the wearer of the drug delivery system, the analyte #2 530 may generate data based a specific protein generated during exercise by a wearer of the drug delivery system. Similarly, the analyte #3 532 and the analyte #4 534 may be configured to generate data related to measurements of analytes which the analyte sensors are specially configured to detect. The data generated by the respective analyte sensors 528-534 may be output via a data connection 536. The electrical connections 536 may receive electrical power from power contacts of a drug delivery device as shown in other examples. Other technical features may be readily apparent to one skilled in the art from the following figures, descriptions, and claims.

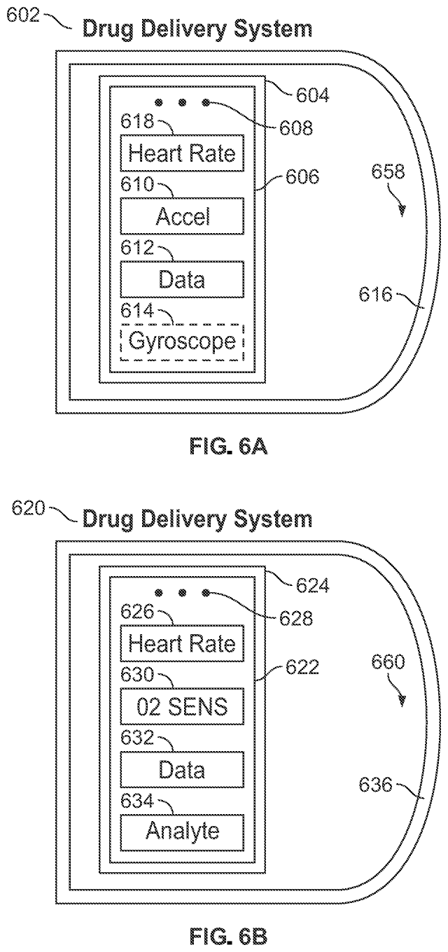

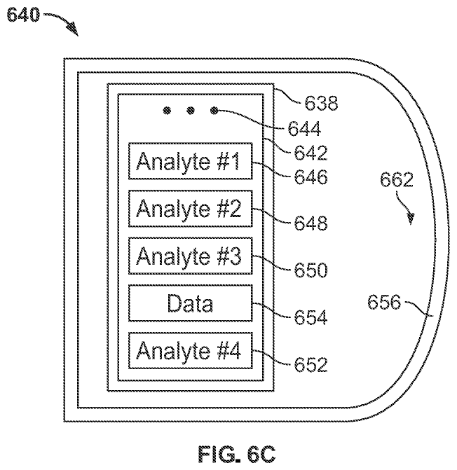

FIG. 6A illustrates a bottom view of an example of a sensor module within a drug delivery device in an aspect of a drug delivery system. In the illustrated example, the sensor module 606 is inserted in a bottom of a drug delivery device 658. The bottom of the drug delivery device 658 may include an adhesive layer 616 that is configured to adhere the drug delivery system 602 to a skin surface of a user.

As described with reference to earlier examples, the sensor module 606 may be coupled either via a mechanical coupling or via a magnetic coupling in the module opening 604 of the drug delivery device 658.

In this example, the drug delivery system 602 includes a drug delivery device 658 and a sensor module 606. The drug delivery device 658 includes a module opening 604 and an adhesive layer 616. The drug delivery device 658 may, as shown in other examples, include an adhesive layer 616 configured to maintain the housing of the drug delivery device 658 in contact with a surface, such the skin of a wearer of the drug delivery system 602. In the example, the sensor module 606 may be held in the module opening 604 via either a mechanical coupling or a magnetic coupling as shown in earlier examples. In the example of FIG. 6A, the sensor module 606 may include a heart rate sensor 618, a three-axis accelerometer 610 and a three-axis gyroscope 614. The heart rate sensor 618 may be a photoplethysmographic sensor configured to make heart rate measurements.

The heart rate sensor 618 (e.g., photoplethysmographic sensor), the accelerometer 610 and the gyroscope 614 may be configured to output signals via a transceiver coupled to the data connection 612 (as described in more detail with reference to another drawing). The data connection 612 may couple to an auxiliary device interface (not shown in this drawing) of the drug delivery device 658. Alternatively, the data connection 612 may be coupled to the accelerometer 610, gyroscope 614 and the heart rate sensor 618 and may couple to a data connection of the drug delivery device 658.

The sensor module 606 when coupled to the drug delivery device 658 may receive power from power contacts 608 from a power source (shown in another example) of the drug delivery device 658.

FIG. 6B illustrates a bottom view of another example of a sensor module within a drug delivery device in an aspect of a drug delivery system. The drug delivery system 620 includes a drug delivery device 660 having a module opening 624 and an adhesive layer 636; and a sensor module 622 having a heart rate sensor 626, an analyte sensor 634, a power contacts 628, a blood oxygen sensor 630 (e.g., like the oxygen saturation sensor 514), and a data connection 632.

In the illustrated example, the sensor module 622 is inserted in a bottom of a drug delivery device 660. The bottom of the drug delivery device 660 includes the adhesive layer 636 that is configured to adhere the drug delivery system 620 to a skin surface of a user (as shown in another drawing).

As described with reference to earlier examples, the sensor module 622 may be coupled either via a mechanical coupling or via a magnetic coupling in the module opening 624 of the drug delivery device 660.

The drug delivery device 658 may, as shown in other examples, include an adhesive layer 636 is configured to maintain the drug delivery device 660 in contact with a surface, such the skin of a wearer of the drug delivery system 620. In the example, the sensor module 622 may be held in the module opening 624 via either a mechanical coupling or a magnetic coupling as shown in earlier examples.

The heart rate sensor 626 may be a photoplethysmographic sensor that is configured to detect a heart rate of the wearer. Similarly, the blood oxygen sensor 630 may configured to detect an oxygen saturation of the wearer. The analyte sensor 634 may be operable to detect an analyte, such as blood glucose, a protein, a drug, a hormone or the like, in the blood of a wearer.

Each of the respective sensors in the sensor module 622 may be configured to output signals via a transceiver coupled to the data connection 632 (as described in more detail with reference to another drawing). Alternatively, the data connection 632 may couple to an auxiliary device interface (not shown in this drawing) of the drug delivery device 660.

The sensor module 622 when coupled to the drug delivery device 660 may receive power from power contacts 628 from a power source (shown in another example) of the drug delivery device 660.

FIG. 6C illustrates a bottom view of yet another example of a sensor module within a drug delivery device in an aspect of a drug delivery system. The drug delivery system 640 includes a sensor module 642 and a drug delivery system 640. The sensor module 642 includes a power contacts 644, an analyte sensor #1 646, an analyte sensor #2 648, an analyte sensor #3 650, an analyte sensor #4 652, and a data connection 654. The analytes detected by the respective analyte sensors 646, 648, 650 and 652 may detect different analytes, such as blood glucose, a protein, a drug, a hormone or the like, in the blood of a wearer. For example, analyte sensor #1 646 may detect blood glucose, analyte sensor #2 648 may detect a protein indicative of exercise, analyte sensor #3 650 may detect the presence of a drug, such as insulin, in the blood of a wearer, and analyte sensor #4 652 may detect the presence of testosterone in the blood of the wearer.

The drug delivery device 662 also includes an adhesive layer 656 that may be configured to secure the drug delivery device 662 and sensor module 642. The sensor module 642 may receive power from power contacts 644 from a power source (shown in another drawing) of drug delivery device 662.

While specific variations of sensors are shown in each of the respective sensor modules illustrated in FIGS. 5A-6C, the combination of sensors in a sensor module is not limited to the illustrated specific variations.

The system comprises a drug delivery system 702, a sensor module 704, a drug delivery device 706, a skin 708, an adhesive layer 710, and a sensor skin access 712.

The system comprises a drug delivery system 714, a sensor module 716, a drug delivery device 718, a skin 720, an adhesive layer 722, and a module opening 724. Once snap fitted, the sensor module may be flush with the rest of the AID system and when placed on the skin provide good contact for the PPG sensor to work effectively. The relatively rigid mounting of the sensor module to the AID system reduces motion artifacts and thereby increases accuracy of measurement for both the PPG sensor as well as the accelerometer and gyroscope. An advantage of the snap fit is that it provides a secure mechanical attachment while establishing electrical contact for power delivery as well as receiving data.

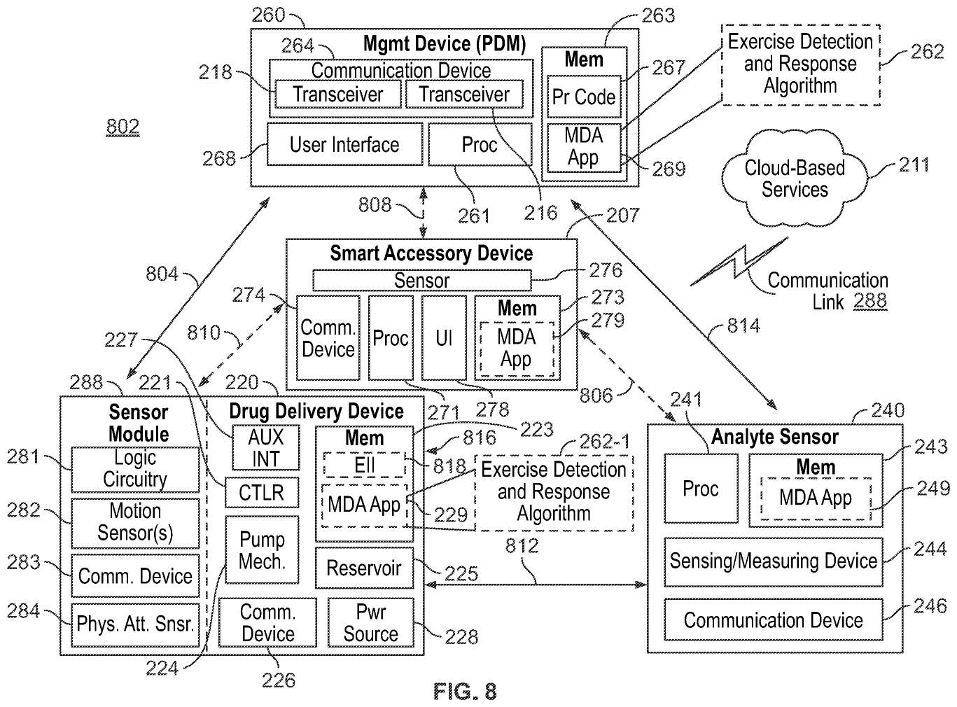

FIG. 8 illustrates a functional block diagram of a system example suitable for implementing the example processes and techniques described herein.

The drug delivery system environment 802 may be include components that may be referred to as an automatic drug delivery system that is configured to deliver a drug without any user interaction, or in some examples, limited user interaction, such as in response to depressing a button to indicate the onset of exercise, or the like.

The drug delivery system environment 802 in some examples may include a management device 260, a drug delivery system 816, an analyte sensor 240, and cloud-based services 211. In another example, the drug delivery system environment 802 may include a management device 260, a drug delivery system 816, an analyte sensor 240, cloud-based services 211 as well as the smart accessory device 207. In yet another example, the drug delivery system environment 802 may include a drug delivery system 816, and an analyte sensor 240. In any of the examples of the drug delivery system environment 802, the cloud-based services 211 as well as the smart accessory device 207 may be optional.

Different systems or devices of the drug delivery system environment 802 may implement (and/or provide functionality for) a medication delivery algorithm or application (MDA). An example of an MDA may be an artificial pancreas (AP) application that may be configured to govern or control automated delivery of a drug or medication, such as insulin, to a user (e.g., to maintain euglycemia—a normal level of glucose in the blood). The MDA may, for example, receive information from additional applications or algorithms that execute on a device within the drug delivery system environment 802.

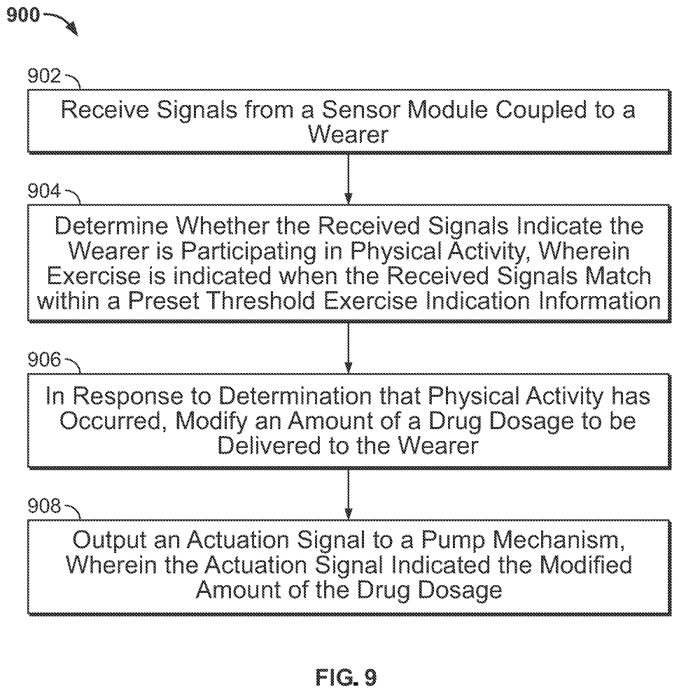

The drug delivery system 816 may include a drug delivery device 220 and a sensor module 288. The drug delivery device 220 may be configured to perform and execute the processes described in the examples of FIG. 9 without input from the management device 260 or the optional smart accessory device 207. The drug delivery device 220, in the example drug delivery system 816, may include an auxiliary interface 227, a controller (CTLR) 221, a pump mechanism 224, a communication device 226, a memory 223, a power source 228, and a reservoir 225.

The sensor module 288 may include logic circuitry 281, a motion sensor(s) 282, a communication device 283, and a physical attribute sensor 284 (labeled as Phys. Att. snsr. in the figure). The logic circuitry 281 The motion sensor(s) 282 may be one sensor or a number of different sensors, such as the accelerometer and the gyroscope as described with reference to earlier figures as well as other different sensors that are operable to detect orientation and movement that may be associated with exercise. The physical attribute sensor 284 may be configured to detect physical attributes of a wearer, such as a heart rate of the wearer, a blood oxygen saturation of the wearer, an analyte (e.g., blood glucose or hormone) of the wearer, a combination of physical attributes using the listed sensors or different sensors. Both the motion sensor 282 and the physical attribute sensor 284 may be configured to output a signal (or multiple signals). The physical attribute sensor 284 may include one or more sensors such as those shown in FIGS. 5A-6C, or a combination of the various sensors shown in the FIGS. 5A-6C. The sensor module 288 may be configured as a fitness device to enable the user to forego wearing a separate fitness device. In this example, the logic circuitry 281 of the sensor module 288 may be configured to generate data like that usually provided by a fitness device, such as a pedometer, calorie usage calculator, or the like.

The logic circuitry 281 may be configured to make the determination of whether the user participated in exercise, a type of the exercise and a category of the exercise, such as aerobic or anaerobic. Alternatively, the controller 221 may be configured to make the determination of whether the user participated in exercise, a type of the exercise and a category of the exercise, such as aerobic or anaerobic. In another alternative, the processor 261 of the management device 260 may be configured to receive signals generated by the sensor module from the drug delivery device 220 and may be further configured to make the determination of whether the user participated in exercise, a type of the exercise and a category of the exercise, such as aerobic or anaerobic. In a further alternative, the partial processing of the sensor data (e.g., accelerometer data or gyroscope data) may occur at the logic circuitry 281, the controller 221 and the processor 261 of the management device 260. In addition, or alternatively, the processor 271 of the smart accessory device 207 may perform some of the processing of the sensor data or may facilitate transfer of the sensor data from the drug delivery system to the management device 260.

In an operational example, the physical attribute sensor 284 may include an accelerometer, a gyroscope and a heart rate monitor (as shown in FIG. 6A). The controller 221 may be further configured to evaluate the different data provided by the respective sensors within the sensor module 288 to further classify the activity of the wearer, process the different data from the respective sensors and provide indications, such as estimates of a number of calories burned, mean heart rate, duration of activity, number of steps and the like, that are commonly provided by fitness-type devices. For example, the controller 221 may be configured when executing the exercise detection and response algorithm to determine based on the data received from the sensor module that the wearer participated in an exercise that included running for a duration of X minutes, and expending calories estimated to be C, where X and C are time and caloric values, respectively). The duration of X minutes may be 60-120 minutes or the like for aerobic exercise, and may be much less, such as 10-15 minutes, for anaerobic exercise. Based on the determinations from the different data provided by the sensors, the exercise detection and response algorithm may cause the presentation of different prompts or statistics based on the determinations on a user interface of the management device.

The controller 221 alone may implement the processes to determine a response to the detection of exercise as described with respect to the other examples, based on inputs from the sensor module 288. The controller 221 of the drug delivery device 220 may be operable to implement delivery of a drug to the user according to a diabetes treatment plan or other drug delivery regimen stored in the memory 223. For example, the controller 221 may be operable to execute programming code and be configured when executing non-transitory programming code of a medication delivery application or algorithm, such as MDA APP 229 and other programs, such as an exercise detection and response algorithm 262, to perform the functions that implement the example routines and processes described herein. In an operational example, the controller 221, when executing the programming code implementing MDA APP 229, may be configured to output a control signal causing actuation of the pump mechanism 224 to deliver drug dosages or the like as described with reference to the example of FIG. 9.

The memory 223 may store programming code executable by the controller 221. The programming code, for example, may enable the controller 221 to control expelling insulin from the reservoir 225 and control the administering of doses of medication based on signals from the MDA APP 229 or, external devices, when the drug delivery device 220 is configured to receive and respond to the external control signals and be operable to deliver a drug based on information received from the analyte sensor 240, the cloud-based services 211 and/or the management device 260 or optional smart accessory device 207. The memory 223 may also be configured to store other data and programming code, such as the exercise detection and response algorithm 262-1.

The reservoir 225 may be configured to store drugs, medications or therapeutic agents suitable for automated delivery, such as insulin, morphine, hormones, glucagon, blood pressure medicines, chemotherapy drugs, or the like.

In an example, the drug delivery device 220 includes a communication device 226, which may be a receiver, a transmitter, or a transceiver that operates according to one or more radio-frequency protocols, such as Bluetooth, Wi-Fi, a near-field communication standard, a cellular standard, or the like. The controller 221 in addition to communicating with the sensor module 288 may, for example, communicate with the management device 260 and an analyte sensor 240 via the communication device 224.

When configured to communicate with an external device, such as the PDM 260 or the analyte sensor 240, the drug delivery device 220 may receive signals over the communication link 804 from the management device 260 or communication link 812 from the analyte sensor 240. The controller 221 of the drug delivery device 220 may receive and process the signals from the respective external devices (e.g., cloud-based services 211, smart accessory device 207, or management device 260) to implement (or modify) delivery of a drug to the wearer in response to signals received from the sensor module 288.

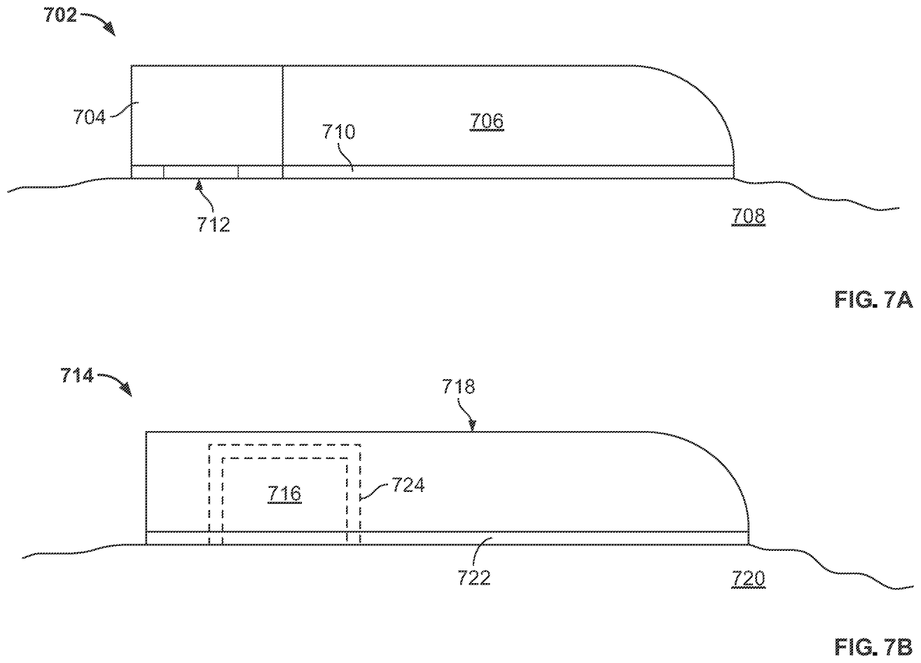

In an operational example, the drug delivery system 816 with the sensor module 288 and the drug delivery device 220 may be coupled to one another as described with respect to FIGS. 1A-7B. The logic circuitry 281 of the sensor module 288 may be integrated circuits (ICs), application specific ICs (ASICs), field programmable arrays (FPGAs), and/or programmable logic devices (PLDs), or the like, that are configured to receive signals from the respective motion sensors 282, communication device 283 and physical attribute sensor(s) 284. If the received signals need processing, the logic circuitry 281 may be configured to process the received signals and output a signal resulting from the processing. Alternatively, if processing is unnecessary, the logic circuitry 281 may be configured to forward the received signals for output. When the sensor module 288 is coupled to the drug delivery device 220, the logic circuitry 281 may be communicatively coupled either via wired couplings (e.g., electrical contacts) to the auxiliary interface 227 or a wireless link, such as Bluetooth, to the communication device 226 that may be communicatively coupled to the auxiliary interface 227 of the drug delivery device 220. The auxiliary interface 227 may be operable to deliver electrical power to a rechargeable battery, such as power source 228. For example, the auxiliary interface 227 may have a port (e.g., mini-USB or the like) to receive a charging cable and wired connections to a rechargeable power source 228 that enable recharging, for example, when the user is sleeping.

In the drug delivery device 220, the auxiliary interface 227 is coupled to the controller 221. The controller 221 is configured to process signals received from the auxiliary interface 227. When the controller 221 executes programming code stored in the memory 223, such the MDA APP 229 and the exercise detection and response algorithm 262, the controller 221 is configured to determine whether a wearer of the drug delivery system 816 is participating in exercise based on the signals received from the sensor module 288 via the auxiliary interface 227. For example, the signals received from the sensor module 288 may indicate an exercise status of a user as well as other information. In an operational example, the controller 221 while executing an instance of the exercise detection and response algorithm 262-1 may access the memory 223 to access exercise indication information, such as signal parameters, that has been confirmed (by the wearer or clinically) to indicate participation in exercise by the wearer. The exercise indication information 818 may be a signal pattern or other parameters (e.g., predetermined maximum signals or the like) that the controller 221 compares to signals received from the logic circuitry 281, and generates an output indicating a result of the comparison.

The exercise indication information 818 may be generated using machine learning techniques. In an example, machine learning classifiers may be trained to classify signals received from the accelerometer and/or gyroscope signals as an activity state of the wearer (e.g., at rest, walking, lying down, running, climbing stairs, aerobic machines (such as an elliptical machine, a rowing machine or a treadmill)). The exercise detection and response algorithm may be configured measure the duration of activity and the heart rate of the wearer, which may be used in conjunction to determine if the wearer is participating in either aerobic or anaerobic exercise.

The determination of whether the wearer is participating in either aerobic or anaerobic exercise is relevant to the controller since the liver produces less glucose when a person (including the wearer) is participating in aerobic activity than when a person (also including the wearer) is participating in anaerobic activity. Based on a result of the determination of the type of exercise (i.e., aerobic versus anaerobic), the controller 221 may modify delivery of the drug to the wearer and calculate a modified dosage and a delivery schedule for the modified dosage or dosages. The current insulin on board may also be factored in when recommending modifications to future insulin delivery.

The controller 221 when executing the MDA APP 229 may output a control signal operable to actuate the pump mechanism 224 to deliver a drug, such as insulin, in response to a determination of a user exercising.

The drug delivery system 816 may be a wearable automatic drug delivery system that may be attached to the body of a user (i.e., a wearer), such as a patient or diabetic, at an attachment location via an adhesive layer (as shown in other figures) and may deliver any therapeutic agent, any drug or medicine, such as insulin or the like, to a user at or around the attachment location.

The drug delivery device 220 may, for example, include a reservoir 225 for storing the drug (such as insulin), a needle or cannula (not shown in this example) for delivering the drug into the body of the user (which may be done subcutaneously, intraperitoneally, or intravenously), and a pump mechanism 224 for transferring the drug from the reservoir 225 through a needle or cannula and into the user. The pump mechanism 224 may be fluidly coupled to reservoir 225, and communicatively coupled to the controller 221.

The drug delivery device 220 may further include a power source 228, such as a battery, a piezoelectric device, other forms of energy harvesting devices, or the like, for supplying electrical power to the pump mechanism 224 and/or other components (such as the controller 221, memory 223, and the communication device 226) as well as to the sensor module 288. Different techniques may be used to obtain harvest energy such as piezoelectric transducers, and the like for storage as electrical energy in the power source 228. The power source 228 may have enough electrical energy storage capability to supply power to the sensor module 288 for its needs.

The power source 228 may be a rechargeable battery or a number of rechargeable batteries. The rechargeable battery 228, the sensor module 288, the drug delivery device 220 or a combination may include a wireless charging receiver interface (not shown) and a secondary charging module (not shown) with a complimentary wireless power transmission interface. For example, the secondary charging module may be operable to interface with an exposed surface or surfaces of the drug delivery device 220 to attach during a recharging process. Attachment to an exposed surface or surfaces of the drug delivery device 220 may be accomplished with magnets, reusable adhesive, mechanical interference fit clamps, or any other method of locating and securing the power transmission interface to the drug delivery device 220. In a further example, the secondary charging module may not have a charging cable and may be operable to mount directly to the drug delivery device 220. In another example, the charging module may have a cable with a specially designed transmitter dongle which connects to an interface, such as the auxiliary interface 227 of the drug delivery device 220.

The secondary charging module in a further example may be rechargeable via an auxiliary cable, such as USB, mini-USB or the like, a wall outlet, an automobile 12V power, a renewable power source or the like. Alternatively, the secondary charging module may use replaceable batteries that may be swapped by the user as needed.

The benefits of the recharging system would allow the infusion device to remain fully sealed even if the rechargeable battery is part of a reusable pump component. Typical configurations would have the user swap reusable components while one charges. Being able to charge in place removes this restriction

The logic circuitry 281 may be configured to draw power only at prescribed times. For example, the logic circuitry 281 may be configured with a clock that may be synchronized to a time of day, such that the logic circuitry 281 may be able to determine when a wearer of the drug delivery system is sleeping. As a result, for purposes of exercise detection, the logic circuitry 281 does not permit power to be delivered to the respective sensors or components of the sensor module 288. Alternatively, the exercise detection and response algorithm 262-1 may be configured, in addition to including programming code enabling functions as an exercise detection or fitness device, to function as a sleep monitor. As a sleep monitor, the controller 221, when executing the exercise detection and response algorithm 262-1, may use the sensor data provided by the respective sensors from a sensor module 288 in the analysis of the wearer's sleep. For example, the accelerometer data or the gyroscope data may be used to indicate how restful a wearer's sleep was for the night. The accelerometer data or the gyroscope data may be compared to patterns or signatures obtained from periods of restful sleep, frenetic sleep or the like as indicated by the wearer.

The exercise detection and response algorithm 262-1 may also have a power management capability that places the sensor module 288 into a sleep mode when activity below a minimum threshold is detected, or the gyroscope data and the acceleration data indicate the wearer is lying down or the like. In response to detecting the activity below the minimum threshold, the controller 221 may transition into a sleep mode, or sleep state. The controller 221 may respond to a spike in the accelerometer signal magnitude by reverting from the sleep mode, or sleep state, to an exercise detection mode.

The smart accessory device 207, may be a device such as a smartwatch, a personal assistant device or the like, which may communicate with the other components of drug delivery system environment 802 via either a wired or wireless communication links 806, 808 or 810. The smart accessory device 207 may be, for example, an Apple Watch®, other wearable smart device, including eyeglasses, provided by other manufacturers, a global positioning system-enabled wearable, a wearable fitness device, smart clothing, or the like. Like the management device 260, the smart accessory device 207 may also be configured to perform various functions including controlling the drug delivery system 816. For example, the smart accessory device 207 may include a communication device 274, a processor 271, a user interface 278, a sensor 276, and a memory 273. The user interface 278 may be a graphical user interface presented on a touchscreen display of the smart accessory device 207. The sensor 276 may include a heart rate sensor, a blood oxygen saturation sensor, an accelerometer, a gyroscope, a combination of these sensors, or the like. The memory 273 may store programming code to operate different functions of the smart accessory device 207 as well as an instance of the MDA APP 279. The processor 271 that may execute programming code, such as the MDA APP 279 for controlling the wearable automatic drug delivery device 816 to implement the FIG. 9 example as described herein.

The management device 260 may be a computing device such as a smart phone, a tablet, a personal diabetes management device, a dedicated diabetes therapy management device, or the like. In an example, the management device (PDM) 260 may include a processor 261, a management device memory 263, a user interface 268, and a communication device 264. The management device 260 may contain analog and/or digital circuitry that may be implemented as a processor 261 for executing processes based on programming code stored in the management device memory 263, such as the MDA algorithm or application (APP) 269, to manage a response to a wearer participating in exercise. The management device 260 may be used to initially set up, adjust settings, and/or control operation of the wearable automatic drug delivery device 220 and/or the analyte sensor 240 as well as the optional smart accessory device 207.

The processor 261 may also be configured to execute programming code stored in the management device memory 263, such as programming code 267 and the MDA APP 269. The MDA APP 269 may

The user interface 268 may be under the control of the processor 261 and be configured to present a graphical user interface that enables the input of a meal announcement, adjust setting selections and the like as described above.

The communication device 264 may include one or more transceivers such as Transceiver 216 and Transceiver 218 and receivers or transmitters that operate according to one or more radio-frequency protocols. In the example, the transceivers 216 and 218 may be a cellular transceiver and a Bluetooth® transceiver, respectively. For example, the transceivers 216 and 218 may be configured to receive and transmit signals containing information usable by the MDA APP 269.

In some examples, the management device 260 may include a user interface 268, respectively, such as a keypad, a touchscreen display, levers, light-emitting diodes, buttons on a housing (shown in another example) of the management device 260, a microphone, a camera, a speaker, a display, or the like, that is configured to allow a user to enter information and allow the management device 260 to output information for presentation to the user (e.g., alarm signals or the like). The user interface 268 may provide inputs, such as a voice input, a gesture (e.g., hand or facial) input to a camera, swipes to a touchscreen, or the like, to processor 261 which the programming code interprets.

The analyte sensor 240 may include a processor 241, a memory 243, a sensing/measuring device 244 and a communication device 246. The analyte sensor 240 may be communicatively coupled to the processor 261 of the management device 260 or controller 221 of the wearable automatic drug delivery device 220. The memory 243 of the analyte sensor 240 may be configured to store information and programming code, such as an instance of the MDA APP 249.

The analyte sensor 240 may be configured to detect multiple different analytes, such as lactate, ketones, uric acid, sodium, potassium, alcohol levels, hormone levels, or the like, and output results of the detections, such as measurement values or the like. The analyte sensor 240 may, in an example, be configured to measure a blood glucose value at a predetermined time interval, such as every 5 minutes, or the like. The communication device 246 of analyte sensor 240 may have circuitry that operates as a transceiver for communicating the measured blood glucose values to the management device 260 over a wireless link 814 or with wearable automatic drug delivery device 220 over the wireless communication link 804. While called an analyte sensor 240, the sensing/measuring device 244 of the analyte sensor 240 may include one or more additional sensing elements, such as a glucose measurement element, a hormone detection element, a heart rate monitor, a pressure sensor, or the like. The processor 241 may include discrete, specialized logic and/or components, an application-specific integrated circuit, a microcontroller or processor that executes software instructions, firmware, programming instructions stored in memory (such as memory 243), or any combination thereof.

Like the controller 221, the processor 241 of the analyte sensor 240 may be operable to perform many functions. For example, the processor 241 may be configured by the programming code stored in the memory 243 to manage the collection and analysis of data detected the sensing and measuring device 244 and deliver the results of the analysis and/or the data to the management device 260, the drug delivery system 816, or both.

Although the analyte sensor 240 is depicted in FIG. 8 as separate from the wearable automatic drug delivery device 220, in various examples, the analyte sensor 240 and wearable automatic drug delivery device 220 may be incorporated into the same unit. That is, in various examples, the sensor 240 may be a part of the wearable automatic drug delivery device 220 and contained within the same housing of the wearable automatic drug delivery device 220 (e.g., the sensor 240 or, only the sensing/measuring device 244 and memory storing related programming code may be positioned within or integrated into, or into one or more components, such as the memory 243 of, the wearable automatic drug delivery device 220). In such an example configuration, the processor 241 may be able to implement the process example of FIG. 9 alone without any external inputs from the management device 260, the cloud-based services 211, the optional smart accessory device 207, or the like.

The communication link 288 that couples the cloud-based services 211 to the respective devices 220, 240, 260 or 207 of system 802 may be a cellular link, a Wi-Fi link, a Bluetooth link, or a combination thereof. Services provided by cloud-based services 211 may include data storage that stores anonymized data, such as blood glucose measurement values, drug delivery history, bolus delivery history, time data, and other forms of data. In addition, the cloud-based services 211 may process the anonymized data from multiple users to provide generalized information related to clinical diabetes-related data and the like.

The wireless communication links 804, 806, 808, 810, 812, 814 and 288 may be any type of wireless link operating using known wireless communication standards or proprietary standards. As an example, the wireless communication links 804, 806, 808, 810, 812, 814 and 288 may provide communication links based on Bluetooth, Zigbee®, Wi-Fi, a near-field communication standard, a cellular standard, or any other wireless protocol via the respective communication devices 264, 226, 246 and 274.

Software related implementations of the techniques described herein, such as the processes examples described with reference to FIG. 9 may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

A controller of the drug delivery device 220 may administer a drug, such as insulin or glucagon, according to a personal diabetes treatment plan for a wearer of a drug delivery system, such as 816. In an example, the controller, such as controller 221, may receive blood glucose measurement values from an analyte sensor 240 or from a physical attribute sensor, such as physical attribute sensor 284, that may be configured to detect blood glucose. In response to the blood glucose measurement values, the controller in the drug delivery device may determine optimal doses of insulin to be delivered and begin administering the doses according to a delivery schedule.

When a wearer of the drug delivery system is participating in exercise and insulin level is high, glucose in the wearer's body is going to be used as energy by the wearer's cells and the liver is not going to generate any additional glucose (particularly in the case of a person with Type I diabetes). As a result, the amount of glucose in the wearer's blood is going to decrease rapidly and as a result, an occurrence of hypoglycemia is possible. If the wearer has too little insulin in their body, the amount of glucose in the user's body may increase to levels that are above a threshold for hyperglycemia.

In step 902, process 900 receives signals from a sensor module coupled to a drug delivery device worn by a wearer, wherein the signals are from an accelerometer, a gyroscope, and a heart rate sensor, the heart rate sensor is configured to detect a heart rate of the wearer.

In step 904, process 900 determines whether the received signals indicate the wearer is participating in physical activity. As described herein, the physical activity referred to herein may be exercise, physical or mental stress, or the like. In the process 900, exercise may be indicated when the received signals match, within a preset threshold, exercise indication information stored in the memory. In the step 904, the process 900 may include accessing exercise indication information stored in a memory. The received signals may be compared to the exercise indication information accessed in the memory. The physical activity may be determined to be exercise in response to a match, within a preset threshold, between the received signals and the accessed exercise indication information. In the example, the preset threshold may be a percentage of a signal magnitude, such as 10%, 15%, 20%, or the like. The preset threshold may be a percentage of a signal magnitude or the like. The exercise detection and response algorithm may be configured to measure the duration of activity and the heart rate of the wearer, which may be used in conjunction to determine if the wearer is participating in either aerobic or anaerobic exercise.

In the example, the controller may have access to user preference data, such as age, weight, gender and the like, that the controller may use in the calculation of different exercise related parameters, such as maximum heart rate, exercise duration (e.g., an amount of time that is considered to be exercise as opposed to a sudden burst of activity, such as running for a bus or lifting groceries), and the like.

For example, the controller may measure a duration of the exercise. Based on the duration, the controller may categorize the exercise as: aerobic when the duration of the exercise is greater than a period of time (e.g., longer than 15 or 20 minutes), or anaerobic exercise when the duration of the exercise is less than or equal to the period time (e.g., <15 or 20 minutes). The respective periods of time may change as the wearer becomes more physically fit or less physically fit.

For aerobic exercise, the controller 221 may determine the wearer is participating in aerobic exercise based on a heart rate that is equal to or greater than 30% of a wearer's maximum heart rate adjusted for age (i.e., a higher heart rate) a longer period of time. The duration of the higher heart rate being maintained for a longer period (or longer duration) of time, such as approximately 15 minutes or the like, may also be considered as an indication of aerobic exercise. While for anaerobic exercise, the controller 221 may determine the wearer is participating in anaerobic exercise based on a heart rate that is equal to or greater than 60% of a wearer's maximum heart rate adjusted for age for a shorter period, or shorter duration, of time, such as approximately 10 minutes or the like. The shorter period or shorter duration of time is less than the longer period or longer duration of time.

Alternatively, the controller 221 executing the exercise detection and response algorithm may generate patterns or signatures from the accelerometer data, the gyroscope data and heart rate data (as well as a blood oxygen sensor, such as 630, or the like) that are indicative of a specific activity, such as running, cycling, boxing, skipping rope, swimming, or the like. The specific activity may be further categorized as either aerobic or anaerobic exercise. The generated patterns or signatures indicative of the specific activity and the categorization of the specific activity may be stored in a memory (e.g., memory 223 of the drug delivery device 220, memory 263 of the management device 260, or via cloud-based services 211) as exercise indication information, such as exercise indication information 818, accessible by the controller 221. The controller 221 may use patterns generated from accelerometer data and/or gyroscope data received from the sensor module during the wearer's participation in activity in the determination of whether the wearer participated in exercise and the type of exercise. The patterns or signatures generated from the accelerometer data and the gyroscope data may be augmented based on inputs from the wearer. For example, the wearer may input into a user interface of a management device that the user is about to ride a stationary bicycle for 30 minutes. The controller executing the exercise detection and response algorithm may generate patterns or a signature from the accelerometer signals and the gyroscope signals.

It should be further noted that drug delivery system may be positioned at different sites on a wearer, such as at their abdomen, upper arm, thighs and the like. As a result, for a given activity, such as riding the stationary bicycle, the signal patterns or signatures generated from the accelerometer data and the gyroscope data by the controller executing the exercise detection and response algorithm may be different based on the positioning of the drug delivery system that includes the sensor module. These different patterns or signatures may be stored in the memory as part of the exercise indication information. The position of the drug delivery system may also be stored in the exercise indication information.

In step 906, process 900, in response to determination that exercise has occurred, modifies an amount of a drug dosage to be delivered to the wearer. The determination of whether the wearer is participating in either aerobic or anaerobic exercise is relevant to the controller since the liver produces less glucose when a person (including the wearer) is participating in aerobic activity than when a person (also including the wearer) is participating in anaerobic activity. The controller 221 may, for example, be configured to access a look up table of responses to the occurrence of the determination of the type of exercise and the duration of the type of exercise. The controller 221 may be configured to cause an automatic response to the determination, generate a dialog with the wearer via a management device, or both. The accelerometer data (e.g., signals) and the gyroscope data (e.g., signals) may be correlated with the data (e.g., signals) from the heart rate sensor to determine the type of exercise.

In addition, the heart rate may remain elevated when the accelerometer signals and the gyroscope signals indicate the exercise has been completed. In this circumstance, an insulin sensitivity parameter may be applied when making the modification to the amount of a drug dosage to be delivered to the wearer.

Based on a result of the determination of the type of exercise (i.e., aerobic versus anaerobic), the controller 221 may modify delivery of the drug to the wearer and calculate a modified dosage and a delivery schedule for the modified dosage or dosages. For example, if the type of exercise is determined to be aerobic activity, the modification made by the controller 221 to the amount of the drug to be delivered may be intended to provide a temporary reduction of basal insulin or a nocturnal basal reduction (for exercise later in the day or in the evening). The temporary reduction of basal insulin may be a predetermined percentage, such as approximately 10%, 15%, 20% or the like. In an example, the temporary reduction may be adaptive based on type of exercise, the time of day of the exercise, the wearer's blood glucose from a previous day, or the like. The nocturnal basal reduction modification reduces the potential for hypoglycemic events while the wearer is sleeping. In addition, the amount of insulin in bolus dosages typically administered in response to meals after the exercise may be reduced for period of time (e.g., 3-5 hours or the like). Alternatively, or in addition, when responding to the determination that the wearer is or has participated in aerobic exercise, the controller 221 may generate an alert to the wearer for carbohydrate consumption during exercise (e.g., a prompt is generated on user interface of a management device.) Alternatively, or in addition, when responding to the determination that the wearer is or has participated in aerobic exercise, the controller 221 may generate an alert to the wearer to optionally sprint at the end of the exercise to encourage the generation of hepatic glucose production (i.e., output of glucose by the liver).

In contrast, if the type of exercise is determined to be anaerobic, the modification to the delivery of the drug made by the controller 221 may be less of a reduction to the basal dosages of insulin as compared to the reduction made in response to the determination of aerobic exercise. The controller 221 may also be configured to calculate an increased amount of insulin to be delivered as a basal dosage due to the liver outputting additional glucose to fuel the wearer's body's cells during the aerobic exercise. In response to the calculated increase in the basal dosage, the controller 221 may cause delivery of an amount of the drug that has been calculated to process the additional glucose output by the liver during (and possible after) the aerobic exercise.

Alternatively, or in addition, when responding to the determination that the wearer is or has participated in anaerobic exercise, the controller 221 may generate an alert to the wearer to finish up their exercise session with a prolonged cool down aerobic activity. Alternatively, or in addition, when responding to the determination that the wearer is or has participated in anaerobic exercise, the controller 221 may generate an alert to the wearer to consume carbohydrates after exercise and, if no hyperglycemia is present with a modified bolus (reduced insulin to carbohydrate ratio).

In step 908, process 900 outputs an actuation signal to a pump mechanism. The actuation signal may indicate the modified amount of the drug dosage that is to be delivered. The pump mechanism may interpret the actuation signal and delivers the modified amount of the drug from the reservoir of the drug delivery device.

Further the insulin delivery recommendations may be individualized based on the wearer's response in the past. Glucose excursion patterns, incidences of hyperglycemia/hypoglycemia during/after exercise in the past may be used to optimize insulin delivery for the future.

Some examples of the disclosed device or processes may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A drug delivery system, comprising:
a drug delivery device including:
a processor,
a memory storing programming code executable by the processor,
a drug container configured to contain a liquid drug,
a pump drive mechanism configured to expel the liquid drug from the drug delivery device, and
an auxiliary device interface coupled to the processor and configured with a data connection; and
a sensor module configured to couple to the auxiliary device interface, the sensor module including:
one or more sensors, wherein the one or more sensors are respectively configured to measure parameters related to an orientation and a movement of the sensor module and a physical attribute of a user; and
output a signal related to the measured parameters to the auxiliary device interface.

2. The system of claim 1, wherein the one or more sensors of the sensor module includes:
an accelerometer;
a gyroscope;
a heart rate sensor; or
a blood oxygen sensor.

3. The system of claim 2, wherein each of the one or more sensors is configured to output a respective signal for receipt by the data connection of the auxiliary device interface.

4. The system of claim 1, wherein the sensor module further comprises:
a data connector coupled to the data connection of the auxiliary device interface.

5. The system of claim 1, wherein the drug delivery device further comprises:
a housing having a top surface and a bottom surface, wherein the bottom surface has an opening configured to hold the sensor module and an adhesive layer configured to maintain the housing in contact with a surface when adhered to the surface, wherein the sensor module when held in the opening contacts the surface, wherein the surface is skin.

6. The system of claim 1, wherein the one or more sensors of the sensor module further comprises:
a photoplethysmographic sensor configured to make a heart rate measurement;
a three-axis accelerometer; and
a three-axis gyroscope, wherein the photoplethysmographic, the accelerometer and the gyroscope are configured to output signals to the auxiliary device interface.

7. The system of claim 1, wherein drug delivery device further comprises:
a rechargeable power supply; and
power supply connectors coupled to the rechargeable power supply; and
the sensor module further comprises:
power supply contacts, wherein the power supply contacts are coupled to the one or more sensors and configured to contact the power supply connectors.

8. The system of claim 1, further comprising:
a housing, wherein the sensor module and the drug delivery device are integrated in the housing and the one or more sensors are distributed among the processor, memory and needle insertion device.

9. The system of claim 1, wherein the drug delivery device and the sensor module are operable to be worn by the user, and the processor is operable when executing the programming code to:
interpret signals received from the one or more sensors via the auxiliary device interface; and
based on an interpretation of the signals received from the one or more sensors indicating that the user is participating in activity, adjust settings for delivery of an amount of the liquid drug from the drug container, or
based on the interpretation of the signals, determine that the user of the drug delivery device and the sensor module is participating in aerobic exercise, and in response to the determination, temporarily reduce basal delivery of the liquid drug.

10. The system of claim 1, wherein:
the memory is configured to store exercise indication information indicating an activity of a user, wherein the exercise indication information is a signal pattern or other parameters indicating exercise; and
the processor is configured to:
interpret signals received from the one or more sensors via the auxiliary device interface;
access the exercise indication information;
compare a pattern obtained from the signal received from the one or more sensors with the signal pattern or other parameters of the exercise indication information; and
determine, based on a result of the comparing, that the pattern indicates aerobic exercise as a category of activity of the user.

11. The system of claim 10, wherein the processor is configured when executing the programming code to:
interpret signals received from the one or more sensors via the auxiliary device interface, wherein the processor is operable to:
access the exercise indication information;
compare a pattern obtained from the signal received from the one or more sensors with a signal pattern or other parameters of the exercise indication information; and
determine, based on a result of the comparing, that the pattern indicates anaerobic exercise as a category of activity of the user;
based on an interpretation of the signals, determine that a wearer of the drug delivery device and the sensor module is participating in anaerobic exercise; and
based on the determination, increase basal delivery of the liquid drug.

12. The system of claim 1, wherein the drug delivery device further comprises:
a communication device coupled to the processor,
wherein the processor is configured when executing the programming code to:
establish a wireless communication link via the communication device with an external device; and
output a generated alert via the communication device for delivery to the external device via the wireless communication link.

13. The system of claim 12, wherein the generated alert is related to aerobic exercise and includes a recommendation, and the recommendation is for the user to:
consume carbohydrates,
sprint after completing the aerobic exercise, or
after completing the aerobic exercise, to consume carbohydrates and administer a modified bolus dosage of the liquid drug based on a reduced liquid drug to carbohydrate ratio.

14. The system of claim 1, wherein the processor is configured when executing the programming code to:
interpret signals received from the one or more sensors via the auxiliary device interface;
based on an interpretation of the signals, determine that the user of the drug delivery device and the sensor module is participating in anaerobic exercise; and
based on the determination, generate an alert related to both the anaerobic exercise and a diabetes treatment plan of the user of the drug delivery device and the sensor module, wherein the alert is:
a recommendation for the user to finish the anaerobic exercise with prolonged aerobic exercise for a cool down period, or
a recommendation for the user after completing the anaerobic exercise to consume carbohydrates and administer a modified bolus dosage of the liquid drug based on a reduced liquid drug to carbohydrate ratio.

15. The system of claim 1, further comprising:
a management device including a management device processor, a management device memory, a communication device and a user interface, wherein the user interface is configured to present information and receive inputs and the management device processor is operable to:
receive alerts from the drug delivery device, the alerts based on measurements received from the one or more sensors.

16. A method, comprising:
receiving signals from a sensor module coupled to a drug delivery device worn by a wearer of the sensor module and the drug delivery device;
determining whether the received signals indicate the wearer is participating in physical activity; and
in response to determination that physical activity has occurred, modifying an amount of a drug dosage to be delivered to the wearer; and
outputting an actuation signal to a pump mechanism, wherein the actuation signal indicated the modified amount of the drug dosage.

17. The method of claim 16, wherein the signals from the sensor module are from an accelerometer, a gyroscope, and a heart rate sensor.

18. The method of claim 16, wherein the physical activity is exercise, which is indicated when the signals received from the sensor module match within a preset threshold exercise indication information retrieved from a memory.

19. The method of claim 16, wherein determining whether the received signals indicate the wearer is participating in physical activity, further comprises:
accessing exercise indication information stored in a memory;
comparing the received signals to the exercise indication information accessed in the memory; and
determining the physical activity is exercise in response to a match, within a preset threshold, between the received signal and the accessed exercise indication information, wherein the preset threshold is a percentage of a signal magnitude.

20. The method of claim 16, further comprises:
measuring a heart rate of a wearer during the physical activity determined to be exercise; and
categorizing the exercise as:
aerobic when the heart rate is equal to or greater than 30% of a wearer's maximum heart rate for a longer period of time, or
anaerobic when the heart rate is equal to or greater than 60% of a wearer's maximum heart rate for a shorter period of time than the longer period of time.
